(19) United States
(12) Patent Application Publication
(10) Pub. No.: US 2024/0382220 A1
(43) Pub. Date: Nov. 21, 2024

(54) ORTHOPEDIC REAMING DEVICES AND METHODS

(71) Applicant: Zimmer, Inc., Warsaw, IN (US)

(72) Inventor: Cody Johnathon Holmes, Warsaw, IN (US)

(21) Appl. No.: 18/658,145

(22) Filed: May 8, 2024

Related U.S. Application Data
(60) Provisional application No. 63/467,150, filed on May 17, 2023.

(51) Int. Cl. A61B 17/16; A61B 17/56; A61B 90/00

(52) U.S. Cl. CPC A61B 17/1684; A61B 90/03; A61B 2017/564; A61B 2090/036

(57) ABSTRACT

A bone cutting instrument for removing a bone during an orthopedic procedure. The instrument optionally including a shaft having a centerline axis and at least partially cannulated at a distal end portion, a stop coupled to the shaft at or adjacent the distal end portion, wherein the stop is configured to contact a surface of the bone and one or more cutting features coupled to the distal end portion and coupled to the stop. The one or more cutting features can be configured to cut the bone with rotation of the one or more cutting features about the centerline axis of the shaft. The stop can set a desired depth for the one or more cutting features to remove the bone.

### CLAIM OF PRIORITY

This application claims the benefit of U.S. Provisional Patent Application Ser. No. 63/467,150, filed on May 17, 2023, the benefit of priority of which is claimed hereby, and which is incorporated by reference herein in its entirety.

### FIELD OF THE DISCLOSURE

The present disclosure relates to orthopedic apparatuses such as reaming tools and a method of use during an arthroplasty procedure.

### BACKGROUND

In the human body, tissue can require repair. Such tissue includes bone, muscles, tendons, ligaments and cartilage. Forceful twisting, trauma or rotation of a joint can tear or otherwise damage tissue. Disease can also necessitate replacement of bone(s) of the joint with one or more prosthetic components. Thus, a surgical repair of the tissue may be required in various circumstances.

Such replacement can require reaming or other cutting and preparation of bone to receive a prosthesis. Reamers, for example, acetabular reamer cups, humeral metaphysis reamers and scapular glenoid reamers are surgical tools which are used in surgery for cutting of bone to prepare the joint to receive the prosthesis. While cutting instruments are known and are generally effective for their intended purposes, cutting instruments can be improved.

### SUMMARY

The present inventor has recognized, among other things, reamer designs that address one or more potential problems that derive from the use of typical reamers. These problems include the potential for excessive bone heating due to a higher revolution per minute (RPM) rate of rotation of the reamer when cutting the bone. Such heating is undesirable and can result in early implant failure and/or lead to necrosis of the bone area being reamed. Additionally, the present inventor has recognized that typical reamers can be hard to manipulate precisely due to the higher RPM used when operating them. Cutting bone with reamers using the higher RPM can result in off-axis cutting, removal of non-uniform and/or undesired amounts of bone, for example.

In view of these and other problems, the present inventor has realized reamers such as for reaming the metaphysis of the humerus or glenoid during a shoulder replacement procedure that can be precisely manipulated to cut bone at lower rotation rate. Reaming can be performed in a single rotation or less than a rotation. This slower rotation rate for the reamer and/or the single or partial rotation of the reamer when cutting can avoid excessive heating of the bone. Furthermore, the slower rotation rate for the reamer and/or the single or partial rotation of the reamer used in cutting can facilitate more precise manipulation of the reamer. This can result in cutting in a more uniform manner to remove a desired amount of bone.

The above discussion is intended to provide an overview of subject matter of the present patent application. It is not intended to provide an exclusive or exhaustive explanation of the invention. The description below is included to provide further information about the present patent application.

To better illustrate the apparatus and methods disclosed herein, a non-limiting list of examples and techniques is provided here:

Example 1 is an apparatus for removing a bone during an orthopedic procedure, optionally comprising: a shaft having a centerline axis and at least partially cannulated at a distal end portion; a stop coupled to the shaft at or adjacent the distal end portion, wherein the stop is configured to contact a surface of the bone; and one or more cutting features coupled to the distal end portion and coupled to the stop, wherein the one or more cutting features are configured to cut the bone with rotation of the one or more cutting features about the centerline axis of the shaft, wherein the stop sets a desired depth for the one or more cutting features to remove the bone.

In Example 2, the subject matter of Example 1 optionally includes, wherein the one or more cutting features comprise a single cutting feature.

In Example 3, the subject matter of Examples 1-2 optionally includes, wherein an opening is formed between the one or more cutting features, the distal end portion and the stop.

In Example 4, the subject matter of Examples 1-3 optionally includes, wherein each of the one or more cutting features has an arcuate profile along an outer distal surface thereof.

In Example 5, the subject matter of Examples 1-4 optionally includes, wherein the stop has one or more passageways therethrough.

In Example 6, the subject matter of Examples 1-5 optionally includes, wherein a proximal end portion of the shaft includes an impactor plate.

In Example 7, the subject matter of Examples 1-6 optionally includes, wherein the one or more cutting features are configured for cutting the bone with an initial plunging insertion of the apparatus into the bone axially along the centerline axis of the shaft.

In Example 8, the subject matter of Examples 1-7 optionally includes, wherein the stop is configured as a flange having one of a solid body or more spokes coupling the flange to the shaft.

In Example 9, the subject matter of Examples 1-8 optionally includes, wherein the distal end portion extends distal of the stop.

In Example 10, the subject matter of Examples 1-9 optionally includes, wherein the one or more cutting features includes between two and four cutting features, inclusive.

Example 11 is an apparatus for removing part of a humerus during an orthopedic shoulder procedure, optionally comprising: a shaft having a centerline axis; a stop extending from a distal end portion of the shaft, wherein the stop is configured to contact a surface of a metaphysis of the humerus; and one or more cutting features coupled to the distal end portion distal of the stop and extending proximally and radially outward from the centerline axis to couple with the stop, wherein the one or more cutting features are configured to cut the metaphysis with rotation of the one or more cutting features about the centerline axis of the shaft.

In Example 12, the subject matter of Example 11 optionally includes, wherein the stop has one or more passageways therethrough, and wherein each of the one or more cutting features has an arcuate profile along an outer distal surface thereof.

In Example 13, the subject matter of Examples 11-12 optionally includes, wherein the one or more cutting features comprise a single cutting feature, and wherein an opening is formed between the one or more cutting features, the distal end portion and the stop.

In Example 14, the subject matter of Examples 11-13 optionally includes, wherein the one or more cutting features are configured for cutting the humerus with an initial plunging insertion of the apparatus into the humerus.

Example 15 is a method of cutting a bone during an orthopedic surgical procedure, the method optionally comprising: implanting a guiding element into the bone; receiving the guiding element with a shaft of a reamer; inserting one or more cutting features of the reamer into the bone; engaging a surface of the bone with a stop; and rotating the shaft a distance of one revolution or less than one revolution about a centerline axis of the shaft to cut the bone with the one or more cutting features.

In Example 16, the subject matter of Example 15 optionally includes, wherein the engaging the surface of the bone with the stop sets a depth for the one or more cutting features.

In Example 17, the subject matter of Examples 15-16 optionally includes, 30 radians per second or less.

In Example 18, the subject matter of Examples 15-17 optionally includes, wherein the inserting the one or more cutting features of the reamer into the bone includes plunging insertion of the one or more cutting features to initially cut the bone.

In Example 19, the subject matter of Examples 15-18 optionally includes, removing the bone with a cutting tool to facilitate the inserting one or more cutting features of the reamer into the bone.

In Example 20, the subject matter of Examples 15-19 optionally includes, passing the cut bone through an opening formed between the one or more cutting features, the shaft and the stop.

In Example 21, the subject matter of Examples 15-20 optionally includes, passing the bone through one or more passages of the stop.

In Example 22, the subject matter of Examples 15-21 optionally includes, wherein the one or more cutting features includes between one and four cutting features, inclusive.

In Example 23, the subject matter of Examples 15-22 optionally includes, wherein the inserting the one or more cutting features of the reamer into the bone includes plunging insertion of a broach to initially cut the bone.

Example 24 is at least one machine-readable medium including instructions that, when executed by processing circuitry, cause the processing circuitry to perform operations to implement of any of Examples 1-23.

Example 25 is an apparatus comprising means to implement of any of Examples 1-23.

Example 26 is a system to implement of any of Examples 1-23.

Example 27 is a method to implement of any of Examples 1-23.

Any of the above examples described herein can include any one or combination of the features disclosed herein.

### BRIEF DESCRIPTION OF THE FIGURES

The above-mentioned and other features and advantages of this disclosure, and the manner of attaining them, will become more apparent and the disclosure itself will be better understood by reference to the following description of examples taken in conjunction with the accompanying drawings, wherein:

FIG. 1 is a side view of a first example of a reamer in the process of being inserted into a metaphysis of the humerus.

FIG. 2 is a cross-sectional view of the reamer of FIG. 1 and further showing a guiding element received by the reamer.

FIG. 3 is a perspective view of the reamer of FIGS. 1 and 2.

FIG. 3A is a plan view of a side of the reamer of FIG. 3.

FIG. 3B is a first enlarged view of a distal end portion the reamer of FIGS. 3 and 3A.

FIG. 3C is a second enlarged view of the distal end portion of the reamer of FIGS. 3 and 3A.

FIG. 4 is a perspective view of a second example of a reamer.

FIGS. 4A and 4B show enlarged views of a distal end portion of the reamer of FIG. 4.

FIG. 5 is a perspective view of a third example of a reamer.

FIG. 5A is a side view of the reamer of FIG. 5.

FIG. 5B is an enlarged plan view of a distal end portion of the reamer of FIG. 5.

FIGS. 6A and 6B are perspective views of an example broach.

FIG. 6C is a side view of the broach of FIGS. 6A and 6B.

FIG. 6D is an enlarged plan view of a distal end portion of the broach of FIGS. 6A and 6B.

FIG. 7 is a diagram of a method of cutting a bone during an orthopedic surgical procedure.

Corresponding reference characters indicate corresponding parts throughout the several views. The exemplifications set out herein illustrate examples of the disclosure, and such exemplifications are not to be construed as limiting the scope of the disclosure any manner.

### DETAILED DESCRIPTION

In describing the examples of the disclosure illustrated and to be described with respect to the drawings, specific terminology will be used for the sake of clarity. However, the disclosure is not intended to be limited to any specific terms or illustrations used herein, and it is to be understood that each specific term includes all technical equivalents.

The present disclosure is directed to a reamer and methods that can be used in joint replacement. The example description relates to the reamer used in a total or partial shoulder arthroplasty. However, the methods and apparatuses disclosed can be used for other bones and other joint repairs. Thus, the concepts of the present application are not limited by the examples provided herein. The bone used herein is not limited to the humerus but can include any applicable bone of the body.

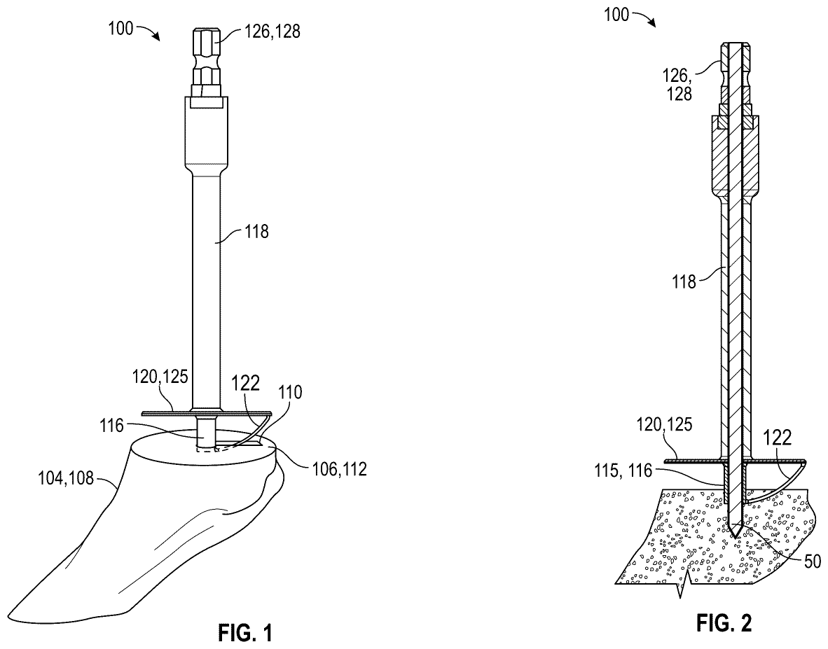

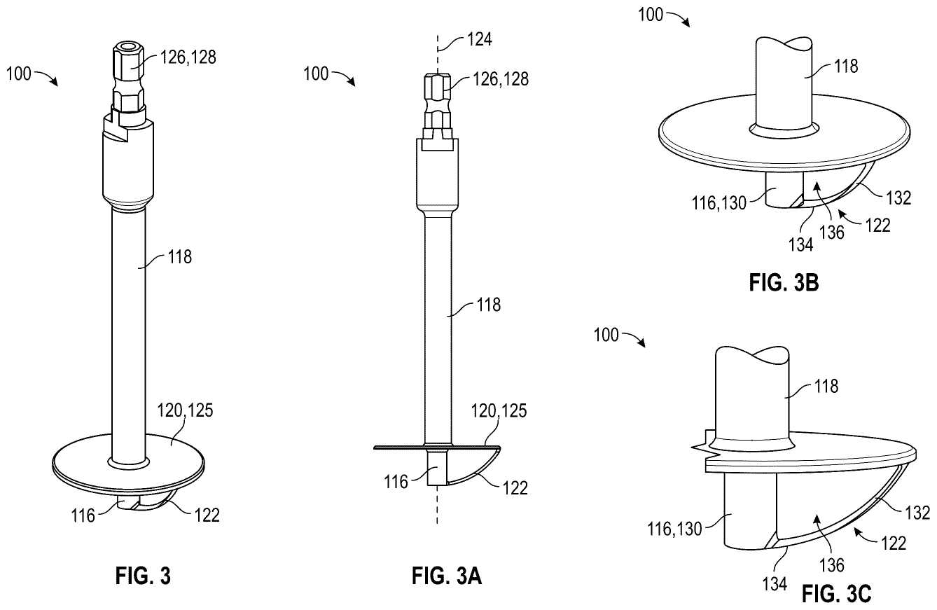

FIGS. 1 and 2 illustrate a first example of a reamer 100 in a process of being inserted into a bone 104 such as a metaphysis 106 of a humerus 108. The process can include preparing the bone such as by resection of a proximal end of the bone 104 and by cutting one or more apertures 110 in a surface 112 of the metaphysis 106 prior to the insertion of the reamer 100 shown in FIGS. 1 and 2. The one or more apertures 110 can be configured (size, shaped, oriented, etc.) to receive one or more cutting features of the reamer 100. The one or more apertures 110 can be formed by a drill or other bone cutting device. The cross-section of FIG. 2 illustrates a guiding element 114 such as a pin, K-wire, bone screw, etc. that can be configured with a sharp tip for penetrating bone as described in U.S. Pat. No. 10,945,862B2, the entire disclosure of which is incorporated herein by reference. The guiding element 114 can be received in a cannulated part 115 (FIG. 2) of a distal end portion 116 of the reamer 100. The guiding element 114 can be used for guiding the reamer 100 to and into the bone 104 at a desired orientation and location, for example.

As shown in FIGS. 1 and 2, the reamer 100 can include a shaft 118, a stop 120 and one or more cutting features 122. The shaft 118 can include the cannulated part 115 (FIG. 2) and the distal end portion 116. The stop 120 can be a flange 125, arm or other projection or feature configured to engage with the surface 112 of the metaphysis 106 as illustrated in FIGS. 1 and 2. The stop 120 can limit the distal insertion depth of the reamer 100, in particular the cut depth of the one or more cutting features 122. This allows a desired amount of the bone 104 to be removed with reaming using the one or more cutting features 122 as further described herein.

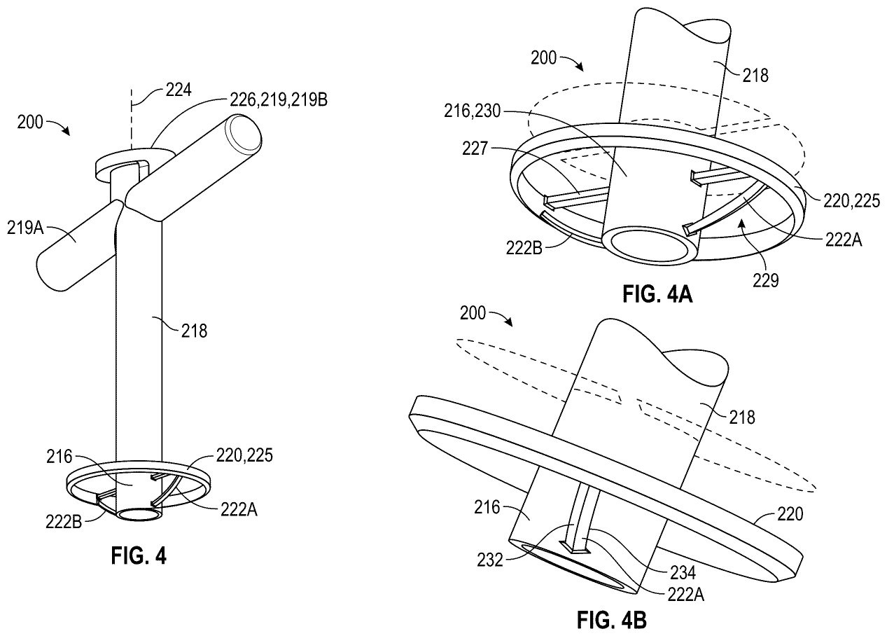

FIGS. 3-3C illustrate the reamer 100 in isolation from the bone and other anatomy of the patient. As shown in FIGS. 3 and 3A, the reamer 100 includes the shaft 118, the stop 120 and the one or more cutting features 122. The stop 120 can be coupled to the shaft 118 at or adjacent the distal end portion 116. The distal end portion 116 can extend distal of the stop 120. The stop 120 can be integrated with (forming a single component with) the shaft 118 or can be attached thereto using known mechanical mechanisms such as welding, mating features or the like. The stop 120 can project radially outward from the shaft 118 as the flange 125. The flange 125 can have a solid body without passageways as shown in FIGS. 3 and 3A.

The one or more cutting features 122 can couple with the stop 120 at a first end (such as at or near an outer edge of the flange 125) and with the distal end portion 116 of the shaft 118 at a second end. The one or more cutting features 122 can be integral with (formed as part of) the shaft 118 and/or the stop 120 or can be a separate component attached thereto via weld, mating attachment or the like. The one or more cutting features 122 are configured to cut the bone 104 with rotation of the one or more cutting features 122 about a centerline axis (described in FIG. 3A below) of the shaft 118.

As shown in FIG. 3A, the shaft 118 can have a centerline axis 124. The shaft 118 can extend along the centerline axis 124 from the distal end portion 116 to a proximal end portion 126. The proximal end portion 126 can be configured as a coupling mechanism 128. For instance, the coupling mechanism 128 can be formed as a male hexagonal projection or a female hexagonal recess for engaging with a male/female part of a driving instrument (not shown), or it can be formed as a bayonet connection for engaging with the driving instrument (not shown). The driving instrument can be a powered driving instrument, such as a power drill, or it can be a manual driving instrument (e.g., a T-handle or a straight handle).

FIGS. 3B and 3C show the distal end portion 116 of the shaft 118 of the reamer 100 in further detail. A nose 130 of the distal end portion 116 can extend distal of the stop 120. The nose 124 can be a necked down or reduced diameter portion of the shaft 118 configured for insertion into the bone such as via the one or more apertures of FIGS. 1 and 2. The nose 130 can include one or more sharps or other bone cutting features for plunging insertion of the nose 130 into the bone in some examples.

FIGS. 3B and 3C show the one or more cutting features 122 are a single cutting feature having an arcuate shape (e.g., an arcuate profile along an outer distal surface 132 and/or the cutting edge thereof). The one or more cutting features 122 can include a sharp edge 134 in the manner of a blade although other examples contemplate other cutting features as known in the art such as teeth, flutes, tines, edges, etc. This sharp edge 134 can be positioned in close proximity to the outer distal surface 132, for example. The arcuate shape of the one or more cutting features 122 can be configured to cut a hemispherical recess in the bone (e.g., the metaphysis). However, other examples for use with other joints and different bones contemplate different shape(s) for the one or more cutting features 122 such as L-shaped, box, rectangular, tapered, conical, etc.

As shown in FIGS. 3B and 3C, an opening 136 can be formed between the one or more cutting features 122, the distal end portion 116 of the shaft 118 (in particular, the nose 130), and the stop 120. The opening 136 can be configured to receive the cut bone after passage of the one or more cutting features 122 distal and radially about of the cut bone with rotation of the cutting features 122 about the centerline axis 124 (FIG. 3A) of the shaft 120.

The reamer 100 of FIGS. 3-3C is configured to cut the bone with the one or more cutting features 122 with rotation of the one or more cutting features 122 about the centerline axis 124 of the shaft 118. This cutting can be performed with one complete (single) rotation of the one or more cutting features 122 according to the example of FIGS. 3-3C. Further rotation of the shaft 118 and the cutting features 122 after the single revolution is unnecessary for cutting the bone. Furthermore, the rotation of the one or more cutting features 122 about the centerline axis 124 of the shaft 118 can be performed at a desired slower rotation rate such as below 30 radians/second. This slower rotation rate can reduce the chances for excessive heating of the bone. Potential side effects such as necrosis can thus be reduced or avoided. Furthermore, slower rotation rate for the reamer and/or single rotation of the reamer can facilitate more precise manipulation of the reamer by the surgeon to perform cutting in a more uniform manner to remove a desired amount of bone.

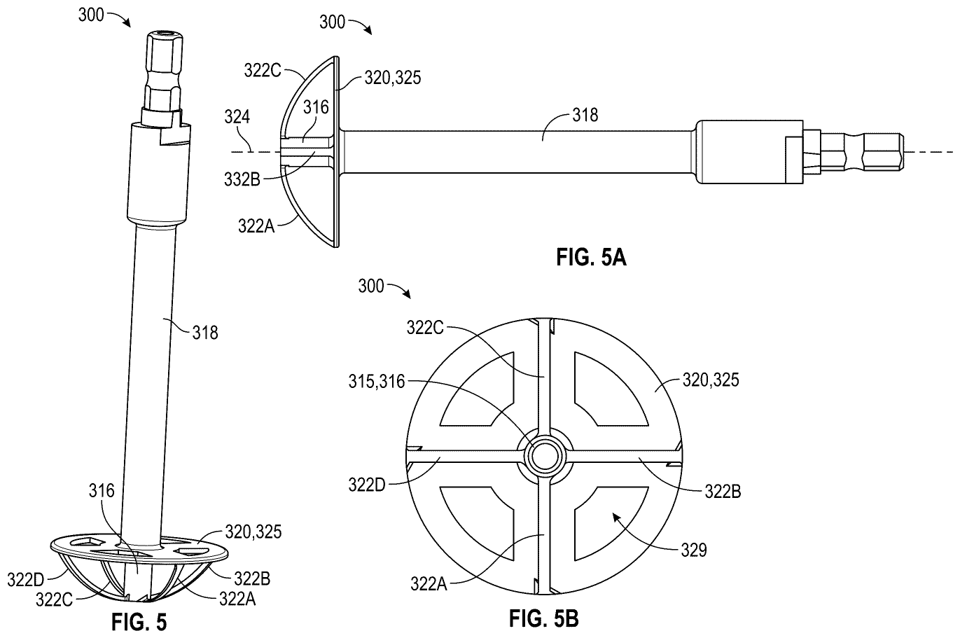

FIGS. 4-4B show a reamer 200 according to another example. As shown in FIG. 4, the reamer 200 can include a shaft 218, a stop 220 and one or more cutting features 222A and 222B. The stop 220 can be coupled to the shaft 218 at or adjacent a distal end portion 216 thereof. A proximal end portion 226 of the shaft 218 can be configured with driving mechanisms 219 such as a T-handle 219A and a striking plate 219B. The reamer 200 can be manually driven for plunging insertion by the surgeon using the striking plate 219B and rotated with the T-handle 219A. The distal end portion 216 can extend distal of the stop 220. The stop 220 can be integral with (forming a single component with) the shaft 218 or can be attached thereto using known mechanical mechanisms such as welding, mating features or the like. The stop 220 can project radially outward from the shaft 218 as a flange 225. The flange 225 can be a rim or ring connected by spokes as further shown in FIGS. 4A and 4B. The one or more cutting features 222A and 222B can couple with the stop 220 at a first end (such as at or near an outer edge of the flange 225) and with the distal end portion 216 of the shaft 118 at a second end. The one or more cutting features 222A and 222B can be integral with (formed as part of) the shaft 218 and/or the stop 220 or can be a separate component attached thereto via weld, mating attachment or the like. The one or more cutting features 222A and 222B are configured to cut bone with rotation of the one or more cutting features 222A and 222B about a centerline axis 224 (FIG. 4) of the shaft 218.

FIGS. 4A and 4B shows the distal end portion 216 of the shaft 218, the stop 220 and the one or more cutting features 222A and 222B (222B is not shown in FIG. 4B). As shown in FIG. 4A, the stop 220 with the flange 225 is attached to the shaft 218 by spokes 227 (FIG. 4A). This configuration for the stop 220 creates one or more passageways 229 through the stop 220. The one or more passageways 229 can be formed between the spokes 227, the shaft 218 and the flange 225. These one or more passageways 229 can allow for the passage of cut bone therethrough. The one or more passageways 229 can also be used by the surgeon to better visualize the cutting of the bone performed with the reamer 200.

Still referring to FIG. 4A, a nose 230 of distal portion 216 can extend distal of the stop 220. The distal portion 216 of the shaft 218 can include a cannulated part 215 for receiving a guiding element as previously described in FIGS. 1-3. The one or more cutting features 222A and 222A are two cutting features on opposing sides of the shaft 218. Each of the one or more cutting features 222A and 222A has an arcuate shape although other shapes are contemplated. As best shown in FIG. 4B, the one or more cutting features 222A and 222A can each have an outer distal cutting edge 232. The distal cutting edge 232 can be configured for cutting the bone with an initial plunging insertion of the reamer 200 into the bone when struck on the striking plate 219B. The one or more cutting features 222A and 222B can include further sharp edges 234 in the manner described previously with regard to the example of FIGS. 3-3C. The sharp edges 234 can be positioned in close proximity to the outer distal cutting edge 232, for example.

The reamer 200 of FIGS. 4-4B is configured to cut the bone with the one or more cutting features 222A and 222A with rotation of the one or more cutting features 222A and 222A about the centerline axis 224 (FIG. 4) of the shaft 218. Cutting can be performed with a partial rotation (about 180 degrees) of the shaft 218 according to the example of FIGS. 4-4B. Further rotation of the shaft 218 and the cutting features 222A and 222A past about 180 degrees is unnecessary for cutting the bone. Furthermore, the rotation of the one or more cutting features 222A and 222A about the centerline axis 224 of the shaft 218 can be performed at a desired slower rotation rate such as below 30 radians/second.

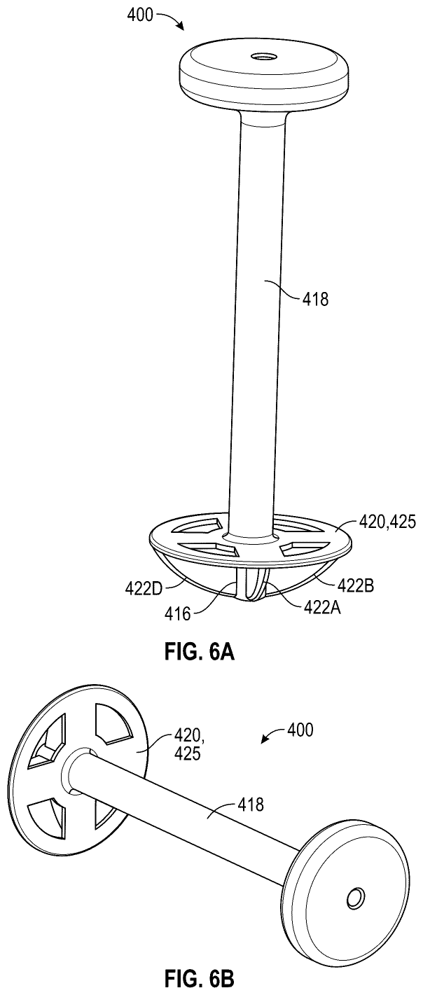

FIGS. 5-5B show a reamer 300 according to yet another example. As shown in FIGS. 5 and 5A, the reamer 300 can include a shaft 318, a stop 320 and one or more cutting features 322A, 322B, 322C and 322D. The shaft 318 can include a distal end portion 316 with a cannulated part 315 (FIG. 5B). The reamer 300 can be constructed with aspects of the reamers 100 and 200 as described previously. The reamer 300 differs from previous the reamers 100 and 200 in that the reamer 300 includes four cutting features 322A, 322B, 322C and 322D. These one or more cutting features 322A, 322B, 322C and 322D can be oriented in 90 degree increments, for example. The stop 320 can be a flange 325 with a plurality of passageways 329 (FIG. 5B) therethrough. The reamer 300 of FIGS. 5-5B is configured to cut the bone with the one or more cutting features 322A, 332B, 322C and 322D with rotation of the one or more cutting features 322A, 322B, 322C and 322D about a centerline axis 324 (FIG. 5A) of the shaft 318. Cutting can be performed with a partial rotation (about 90 degrees) of the shaft 318 according to the example of FIGS. 5-5B. Cutting of bone by the reamer 300 can be performed at the relatively slower speed (e.g., 30 radians/second or less) discussed previously.

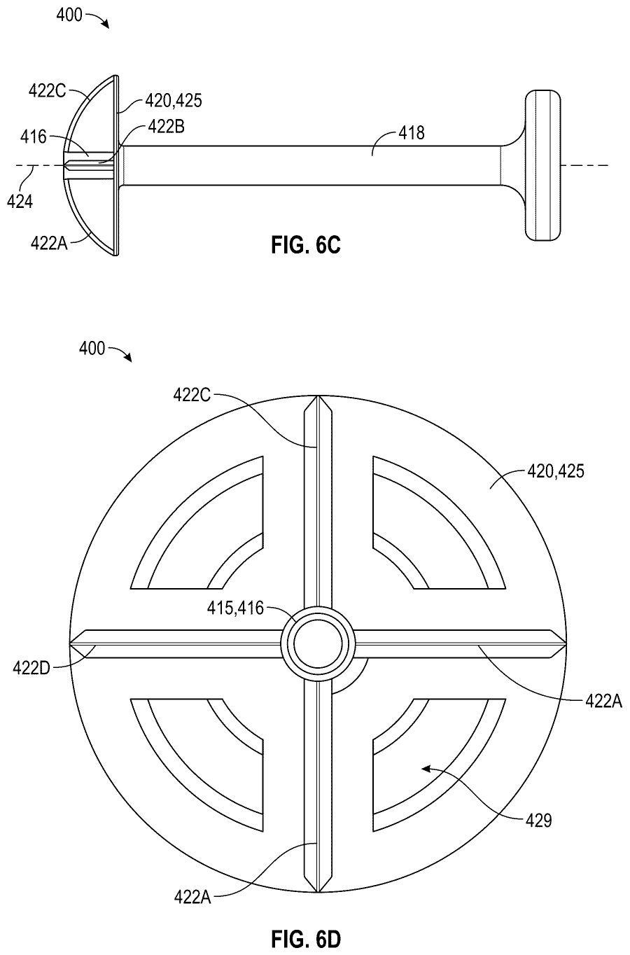

FIGS. 6A-6D show a broach 400. As shown in FIGS. 6A-6C, the broach 400 can include a shaft 418, a stop 420 and one or more cutting features 422A, 422B, 422C and 422D. The shaft 418 can include a distal end portion 416 with a cannulated part 415 (FIG. 6D). The broach 400 can be constructed with aspects of the reamers 100, 200 and 300 as described previously but can be used for making an initial cut such as slot 110 (FIG. 1) in the bone for the reamer. As an example, the broach 400 can be used prior to use of the reamer 300 of FIGS. 5-5B to prepare the bone to receive the reamer 300. The broach 400 differs from previous the reamers 100, 200 and 300 in that the broach 400 includes four cutting features 422A, 422B, 422C and 422D that do not form openings between the cutting features 422A, 422B, 422C and 422D, the shaft 418 and the stop 420. The cutting features 422A, 422B, 422C and 422D are rather solid in a depth dimension rather than having openings like those of the one or more cutting features of FIGS. 1-5B.

The broach 400 of FIGS. 6A-6D is configured to cut the bone with the one or more cutting features 422A, 422B, 422C and 422D. The cutting features 422A, 422B, 422C and 422D can be configured for cutting bone with an initial plunging insertion of the broach 400 into the bone similar to the reamer 200 described in reference to the example of FIGS. 4-4B. This can create recesses (such as one or more apertures 110 of FIG. 1) in the bone to receive a reamer. Additionally, the cutting features 422A, 422B, 422C and 422D can be oriented in 90 degree increments similar to the cutting features 322A, 322B, 322C and 322D of FIGS. 5-5B described previously. The stop 420 can be a flange 425 with a plurality of passageways 429 (FIG. 6D) therethrough for visualization.

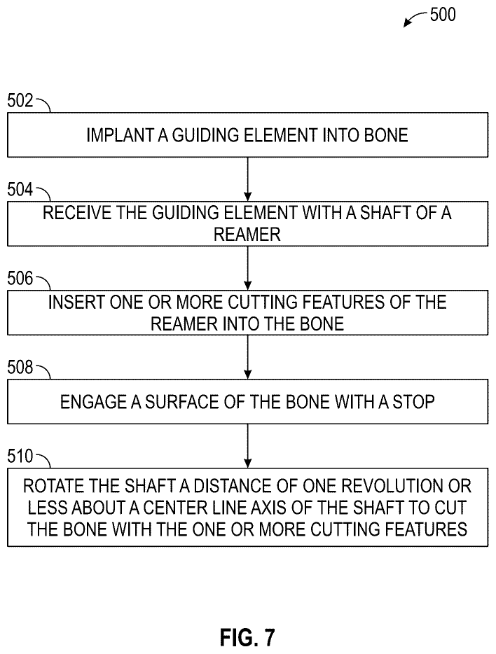

FIG. 7 shows a method 500 of cutting a bone during an orthopedic surgical procedure. The method 500 can include implanting, at step 502, a guiding element into the bone and receiving, at step 504, the guiding element with a shaft of a reamer (see example described in reference to FIGS. 1 and 2). The method 500 can include, at step 506, inserting one or more cutting features of the reamer into the bone. The method 500 can include engaging, at step 508, a surface of the bone with a stop. At step 510, the method 500 can include rotating the shaft a distance of one revolution or less than one revolution about a centerline axis of the shaft to cut the bone with the one or more cutting features.

The one or more cutting features includes between one and four cutting features, inclusive. The engaging the surface of the bone with the stop can set a depth for the one or more cutting features. Rotating the shaft can be at a rate of 30 radians per second or less. The inserting the one or more cutting features of the reamer into the bone can include plunging insertion of the one or more cutting features to initially cut the bone. Alternatively, the method can include preparing the bone by removing the bone with a cutting tool (such as a drill as described in the example of FIGS. 1 and 2) to facilitate the inserting one or more cutting features of the reamer into the bone. The method can include passing the cut bone through an opening formed between the one or more cutting features, the shaft and the stop. The method can include passing the bone through one or more passages of the stop.

Although described in reference to reamers, the principles and designs discussed herein are applicable to other bone cutting tools such as broaches, drills, cannulated drills, needles, osteotomes, rongeurs, bone cutters, punches, etc. are also contemplated that could benefit from the construction of described herein. Thus, the term reamer should not be interpreted as limiting.

The term “proximal” refers to the general orientation of the side and/or surface when the cutting instrument is utilized to cut tissue. Thus, “proximal” refers to a direction or location generally in the direction of or toward a driver, and “distal” refers to the opposite direction of proximal, i.e., away from the driver. As used herein, the terms “anterior” and “posterior” should be given their generally understood anatomical interpretation. Thus, “posterior” refers to a location or direction generally toward a rear of the patient. Similarly, “anterior” refers to a location or direction generally toward a front of the patient. Thus, “posterior” refers to the opposite direction of “anterior.” Similarly, the terms “medial” and “lateral” should be given their generally understood anatomical interpretation. “Medial” refers to the more inward facing (inner part) of the guide or instrument (when in the implanted orientation) and “lateral” refers to the outer part or outward facing part. “Medial” refers to the opposite direction of “lateral.”

It will be readily understood to those skilled in the art that various other changes in the details, material, and arrangements of the parts and method stages which have been described and illustrated in order to explain the nature of the inventive subject matter can be made without departing from the principles and scope of the inventive subject matter as expressed in the subjoined claims. For example, the order of method steps or stages can be altered from that described above, as would be appreciated by a person of skill in the art.

It will also be appreciated that the various dependent claims, examples, and the features set forth therein can be combined in different ways than presented above and/or in the initial claims. For instance, any feature(s) from the above examples can be shared with others of the described examples, and/or a feature(s) from a particular dependent claim may be shared with another dependent or independent claim, in combinations that would be understood by a person of skill in the art.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72 (b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

What is claimed is:

1. An apparatus for removing a bone during an orthopedic procedure, comprising:
a shaft having a centerline axis and at least partially cannulated at a distal end portion;
a stop coupled to the shaft at or adjacent the distal end portion, wherein the stop is configured to contact a surface of the bone; and
one or more cutting features coupled to the distal end portion and coupled to the stop, wherein the one or more cutting features are configured to cut the bone with rotation of the one or more cutting features about the centerline axis of the shaft, wherein the stop sets a desired depth for the one or more cutting features to remove the bone.

2. The apparatus of claim 1, wherein the one or more cutting features comprise a single cutting feature.

3. The apparatus of claim 1, wherein an opening is formed between the one or more cutting features, the distal end portion and the stop.

4. The apparatus of claim 1, wherein each of the one or more cutting features has an arcuate profile along an outer distal surface thereof.

5. The apparatus of claim 1, wherein the stop has one or more passageways therethrough.

6. The apparatus of claim 1, wherein a proximal end portion of the shaft includes an impactor plate.

7. The apparatus of claim 1, wherein the one or more cutting features are configured for cutting the bone with an initial plunging insertion of the apparatus into the bone axially along the centerline axis of the shaft.

8. The apparatus of claim 1, wherein the stop is configured as a flange having one of a solid body or more spokes coupling the flange to the shaft.

9. The apparatus of claim 1, wherein the distal end portion extends distal of the stop.

10. The apparatus of claim 1, wherein the one or more cutting features includes between two and four cutting features, inclusive.

11. An apparatus for removing part of a humerus during an orthopedic shoulder procedure, comprising:
a shaft having a centerline axis;
a stop extending from a distal end portion of the shaft, wherein the stop is configured to contact a surface of a metaphysis of the humerus; and
one or more cutting features coupled to the distal end portion distal of the stop and extending proximally and radially outward from the centerline axis to couple with the stop, wherein the one or more cutting features are configured to cut the metaphysis with rotation of the one or more cutting features about the centerline axis of the shaft.

12. The apparatus of claim 11, wherein the stop has one or more passageways therethrough, and wherein each of the one or more cutting features has an arcuate profile along an outer distal surface thereof.

13. The apparatus of claim 11, wherein the one or more cutting features comprise a single cutting feature, and wherein an opening is formed between the one or more cutting features, the distal end portion and the stop.

14. The apparatus of claim 11, wherein the one or more cutting features are configured for cutting the humerus with an initial plunging insertion of the apparatus into the humerus.

15. A method of cutting a bone during an orthopedic surgical procedure, the method comprising:
implanting a guiding element into the bone;
receiving the guiding element with a shaft of a reamer;
inserting one or more cutting features of the reamer into the bone;
engaging a surface of the bone with a stop; and
rotating the shaft a distance of one revolution or less than one revolution about a centerline axis of the shaft to cut the bone with the one or more cutting features.

16. The method of claim 15, wherein the engaging the surface of the bone with the stop sets a depth for the one or more cutting features, wherein rotating the shaft is at 30 radians per second or less.

17. The method of claim 15, wherein the inserting the one or more cutting features of the reamer into the bone includes plunging insertion of the one or more cutting features to initially cut the bone.

18. The method of claim 15, further comprising removing the bone with a cutting tool to facilitate the inserting one or more cutting features of the reamer into the bone.

19. The method of claim 15, further comprising passing the cut bone through an opening formed between the one or more cutting features, the shaft and the stop.

20. The method of claim 15, further comprising passing the bone through one or more passages of the stop.
